(19) United States
(12) Patent Application Publication
(10) Pub. No.: US 2017/0296352 A1
(43) Pub. Date: Oct. 19, 2017

(54) INTERBODY FUSION CAGE WITH ADJUSTABLE COVER, AND RELATED MANUFACTURE METHOD

(71) Applicant: Yellowsteps, Decines-charpieu (FR)

(72) Inventors: Pierre Dominique Richerme, Praz Sur Arly (FR); Christophe Tamburini, Saint Laurent De Mure (FR)

(73) Assignee: Yellowsteps, Decines-charpieu (FR)

(21) Appl. No.: 15/515,643

(22) PCT Filed: Sep. 30, 2015
(86) PCT No.: PCT/FR2015/052624

§ 371 Date: Mar. 30, 2017

(30) Foreign Application Priority Data
Sep. 30, 2014 (FR) FR 1459304

(51) Int. Cl. A61F 2/44; A61F 2/30

(52) U.S. Cl. CPC A61F 2/4425; A61F 2/447; A61F 2002/30985; A61F 2002/3097; A61F 2002/30784; A61F 2002/30777; A61F 2002/30772; A61F 2/3094; A61F 2002/30538; A61F 2002/30515; A61F 2002/30471; A61F 2002/30266; A61F 2002/4475; A61F 2002/30579

(57) ABSTRACT

The invention concerns an expandable intersomatic cage (1) intended to be implanted between a first and second vertebral bodies of a patient, comprising:
a cage body (2), comprising a bearing surface (6A) intended to be positioned so as to bear against the first vertebral body (4),
an expansion cap (3) mounted on the cage body (2) so as to be able to pivot relative to the bearing surface (6A), said expansion cap (3) comprising an element (3A) for bearing against the second vertebral body (5),
a means (16) for controlling the inclination of the expansion cap (3).
said cage (1) being characterized in that the body (2) comprises at least one fastening orifice (41) forming an oblique well (42) capable of receiving and guiding from the outside of said cage (1) a means for fastening the cage (1) to the first and/or to the second vertebral body.
Surgical implants.

### TECHNICAL FIELD

The present invention relates to the technical field of the surgical implants intended to be implanted in the body of a patient, and in particular in one of the intersomatic spaces of the spine.

In particular, the present invention concerns an expandable or adjustable intersomatic cage intended to be implanted between a first vertebral body and a second vertebral body of a patient, said cage comprising:
a cage body, comprising a bearing surface intended to be positioned so as to bear against the first vertebral body,
an expansion cap, comprising an element for bearing against the second vertebral body, and mounted on the cage body via a linking element so as to be able to pivot according to an inclination stroke about an axis of inclination relative to the bearing surface,
a means for controlling the inclination of the expansion cap capable of maintaining the expansion cap in a desired inclination of the inclination stroke.

The present invention also concerns a method for manufacturing an expandable intersomatic cage intended to be implanted between a first vertebral body and a second vertebral body of a patient, the method including a step during which:
a cage body comprising a bearing surface intended to be positioned so as to bear against the first vertebral body is made, an expansion cap comprising an element for bearing against the second vertebral body is made,
the expansion cap is mounted on the cage body via a linking element, so that the expansion cap could pivot according to an inclination stroke about an axis of inclination relative to the bearing surface,
a means for controlling the inclination of the expansion cap capable of maintaining the expansion cap in a desired inclination of the inclination stroke is made.

### PRIOR ART

These implants aim to treat various pathologies of the spine such as vertebral compression fractures, scolioses, lordoses, kyphoses or vertebral instabilities. Of course, these pathologies are mentioned for an illustrative and non-restrictive purpose.

In particular, these implants are in the form of intersomatic cages. These consist of hollow implants intended to accommodate an osteoinductive material, such as a bone graft. Such cages are intended to be implanted by surgery in the vertebral intersomatic space of a patient, after an adequate preparation of the latter, for example in order to reestablish an unbalanced discal height for the correction of a lordosis that a patient suffers from.

In order to enable the reestablishment of the discal height, certain cages simply have a contact surface with a first spinal segment, and an opposite contact surface with a second spinal implant, said surfaces being inclined with respect to each other, which allows inducing a reorientation of said spinal segments with respect to each other. Nonetheless, in this case, it is necessary to provide a cage the shape of which corresponds accurately to the correction that is desired to be brought.

Thus, expandable cages which allow the surgeon to set the clearance between the contact surfaces have been made, in order to customize the correction that is brought depending on the considered case, and in particular according to the size of the intersomatic space of the patient. In particular, such expandable cages include a fixed element carrying one of the two contact surfaces, as well as a movable element carrying the other contact surface. In general, the movable element is formed by a screw body mounted on a threaded orifice mutual with the fixed element, so that screwing or unscrewing of the movable element allows setting its height.

Nonetheless, such a design requires the surgeon to set the height of the cage before implanting the latter in the body of the patient, and therefore to accurately estimate the dimensions of the space that said cage will have to occupy in order to treat the pathology. Also, once implanted, the height of the cage can no longer be modified, which does not therefore allow the surgeon to modify the correction without having to remove said implant.

In addition, if the height of this cage could be set, for example, in order to reestablish a discal height, said cage does not offer the possibility of setting the inclination of one of the contact surfaces with respect to the other, so that the magnitude of the correction of a lordosis, scoliosis or kyphosis angle cannot be set. Furthermore, in general, the known expandable cages are maintained in position in the body of the patient only by the compression force exerted thereon by the first and second vertebral bodies between which they are inserted. Hence, expandable cages have been made, whose respective bearing surfaces on the first and second vertebral bodies are structured or specially treated so as to contribute to the immobilization of these cages or cages filled with an osteoinductive material intended to spread out of the cage in order to ensure fastening to the vertebral bodies by bone fusion. However, there is still a considerable risk of displacement of such cages for example under the effect of a violent force performed by the patient or repeated vibrations, in particular when the bone fusion is still taking place thanks to the osteoinductive material.

Considering the foregoing, it seems that the implants of the prior art described hereinbefore may be improved.

### DISCLOSURE OF THE INVENTION

Consequently, the objects assigned to the invention aim to remedy to the different aforementioned drawbacks and to propose a new expandable intersomatic cage and a new method for manufacturing an expandable intersomatic cage allowing a set-up of the cage in an accurate, reliable, and modifiable manner and an easy and robust fastening of said cage in the body of the patient.

Another object assigned to the invention aims to propose a new intersomatic cage and a new method for manufacturing an intersomatic cage allowing facilitating the set-up of said cage in the body of the patient and allowing adapting its configuration in situ so as to treat a pathology in a satisfactory manner.

Another object assigned to the invention aims to propose a new intersomatic cage and a new method for manufacturing an intersomatic cage which can be easily set and adapted after its set-up, so as to improve the quality of the treatment.

Another object assigned to the invention aims to propose a new intersomatic cage and a new method for manufacturing an intersomatic cage which is particularly robust and durable.

Another object assigned to the invention aims to propose a new intersomatic cage and a new method for manufacturing an intersomatic cage allowing a rapid treatment of a pathology that a patient suffers from, and in the body of whom said cage is introduced.

Another object assigned to the invention aims to propose a new intersomatic cage and a new method for manufacturing an intersomatic cage the set-up of which in the body of a patient is barely invasive.

Another object assigned to the invention aims to propose a new intersomatic cage and a new method for manufacturing an intersomatic cage which is easy and relatively inexpensive to manufacture.

Another object assigned to the invention aims to propose a new intersomatic cage and a new method for manufacturing an intersomatic cage the set-up and fastening of which in the body of a patient are sure and accurate.

The objects assigned to the invention are achieved by means of an expandable intersomatic cage intended to be implanted between a first vertebral body and a second vertebral body of a patient, said cage comprising:
a cage body, comprising a bearing surface intended to be positioned so as to bear against the first vertebral body,
an expansion cap, comprising an element for bearing against the second vertebral body, and mounted on the cage body via a linking element so as to be able to pivot according to an inclination stroke about an axis of inclination relative to the bearing surface,
a means for controlling the inclination of the expansion cap capable of maintaining the expansion cap in a desired inclination of the inclination stroke,

said cage being characterized in that said cage body comprises at least one fastening orifice forming an oblique well capable of receiving and guiding from the outside of said cage a means for fastening the cage to the first vertebral body and/or to the second vertebral body.

The objects assigned to the invention are also achieved by means of a method for manufacturing an expandable intersomatic cage intended to be implanted between a first vertebral body and a second vertebral body of a patient, the method including a step during which:
a cage body comprising a bearing surface intended to be positioned so as to bear against the first vertebral body is made, an expansion cap comprising an element for bearing against the second vertebral body is made,
the expansion cap is mounted on the cage body via a linking element, so that the expansion cap could pivot according to an inclination stroke about an axis of inclination relative to the bearing surface,
a means for controlling the inclination of the expansion cap capable of maintaining the expansion cap in a desired inclination of the inclination stroke is made.

said method being characterized in that it also comprises a step during which said cage body is provided with at least one fastening orifice forming an oblique well capable of receiving and guiding from the outside of said cage a means for fastening the cage to the first vertebral body and/or to the second vertebral body.

### BRIEF DESCRIPTION OF THE DRAWINGS

Other objects and advantages of the invention will appear better upon reading the description hereinafter, as well as with reference to the appended drawings, provided only for an illustrative and non-restrictive purpose, and in which:

FIG. 1 illustrates according to a lateral schematic view (that is to say according to a direction orthogonal to the anatomical sagittal plane) an example of an intersomatic cage according to the invention, which is implanted in this instance between a first vertebral body and a second vertebral body and comprises an expansion cap which has a minimum inclination, said cage being in accordance with a variant according to which it is provided with one single fastening orifice.

FIG. 2 represents according to a lateral schematic view (that is to say according to a direction orthogonal to the anatomical sagittal plane) the cage of FIG. 1, the expansion cap of which has a maximum inclination in order to modify the relative position of the second vertebral body with respect to the first vertebral body.

FIG. 3 illustrates, according to a top perspective view, the expansion cap of FIGS. 1 and 2.

FIG. 4 illustrates, according to an exploded perspective view, the intersomatic cage of FIGS. 1 and 2 and different elements comprised thereby.

FIGS. 5 and 6 both illustrate, according to a perspective view, a cage body belonging to a cage in accordance with the invention, according to a variant in which said cage is provided with two fastening orifices, and according to an embodiment in which the support and front walls are provided with a plurality of pores and apertures.

FIG. 7 illustrates, according to a perspective view, the cage body of FIGS. 5 and 6, according to an embodiment in which the support and front walls and a lateral wall are provided with a plurality of pores and/or apertures.

FIG. 8 illustrates, according to a perspective view, the cage body of FIGS. 5 and 6, according to an embodiment in which the support and front walls and the bottom plate are provided with a plurality of apertures.

FIGS. 9 and 10 illustrate, according to a front perspective view, a cage including the cage body of FIG. 7, respectively in the minimum (FIG. 9) and maximum (FIG. 10) inclination positions, so as to particularly highlight the configuration of the secondary fastening hole allowing the passage of a fastening means, for example a screw, through the expansion cap.

FIGS. 11 and 12 illustrate, according to a front view, a cage including the cage body of FIG. 8, respectively in the minimum (FIG. 11) and maximum (FIG. 12) inclination positions, according to a variant in which said cage is provided with two fastening orifices and with two screw-type fastening means.

FIGS. 13 and 14 illustrate, according to a perspective view, the cage of FIGS. 11 and 12, respectively in the minimum (FIG. 13) and maximum (FIG. 14) inclination positions, so as to particularly highlight the configuration of the secondary fastening hole allowing the passage of a fastening means through the expansion cap.

FIGS. 15 and 16 illustrate a preferred example of lateral implementation of a cage in accordance with the invention between a first and a second vertebral bodies.

### BEST MODE FOR CARRYING OUT THE INVENTION

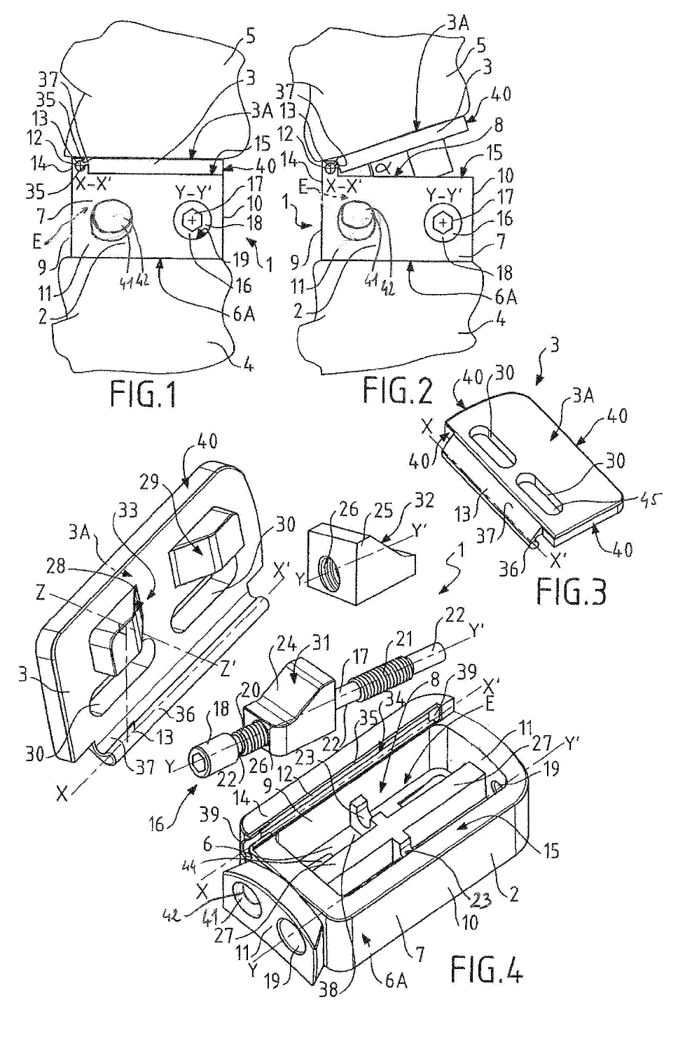

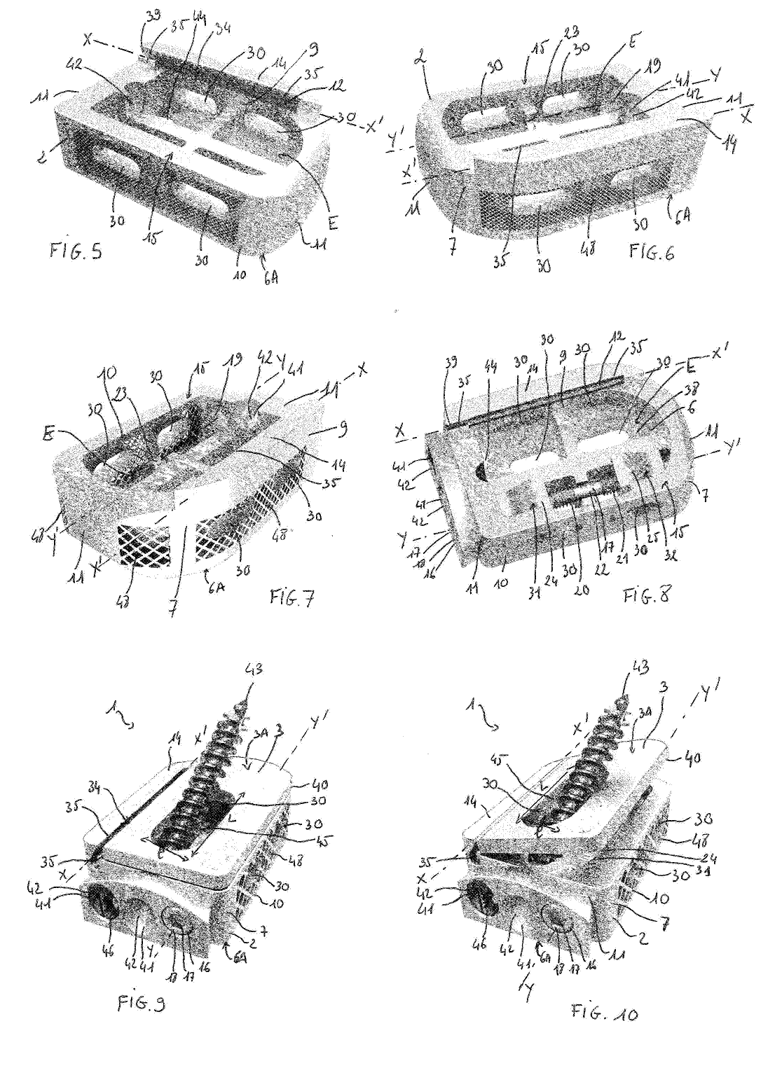

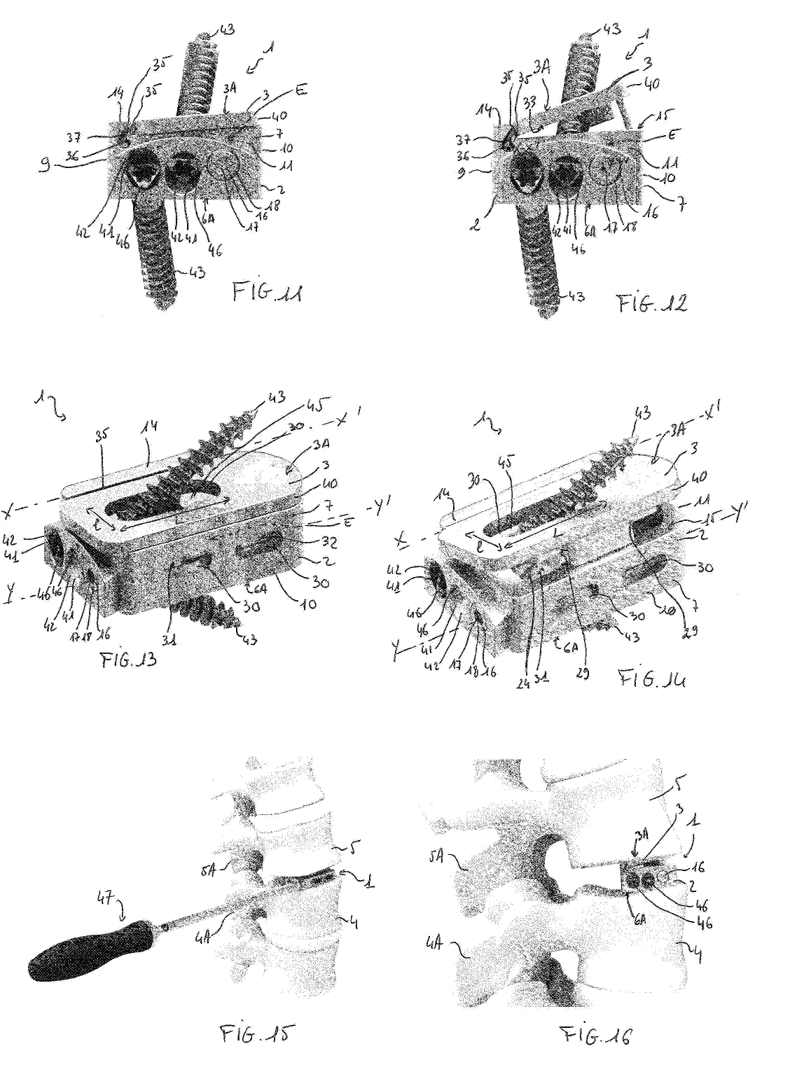

Examples of an expandable intersomatic cage 1 in accordance with the invention are represented in FIGS. 1 to 14. When said cage 1 is implanted in the body of a patient at the level of the spine of the latter, it allows in particular treating different types of pathologies relating for example to a bad orientation of vertebral bodies of the spine with respect to each other, such pathologies including lordoses, scolioses, kyphoses, or vertebral compression fractures.

Preferably, the patient referred to by the invention is a human being, bearing in mind of course that nothing precludes the introduction and fastening of said cage of the invention in the body of an animal in order to heal veterinary pathologies similar to the human pathologies described hereinabove.

The expandable intersomatic cage 1 of the invention is intended to be implanted between a first vertebral body 4 and a second vertebral body 5, and in particular between the vertebral endplates of the latter, so that said cage 1 occupies all or part of the intersomatic space separating said vertebral bodies. The first vertebral body 4 and the second vertebral body 5 being thus separated by the cage 1, the latter advantageously allows positioning them with respect to each other, or at least orientating them with respect to each other, one of said vertebral bodies resting on the other via said cage 1, which forms a wedge.

For example, the first vertebral body 4 and the second vertebral body 5 may form vertebrae, lumbar vertebrae, and/or cervical vertebrae, that is to say, advantageously, bony and/or cartilaginous bodies of the spine of the patient. Preferably, the cage 1 is implantable inside the body of the patient, between the first vertebral body 4 and the second vertebral body 5, during a surgery, performed for example under anesthesia. Advantageously, the intersomatic space intended to accommodate the cage 1 is prepared accordingly, certain corporal elements, such as for example the intervertebral disc, being for example removed.

Preferably, the intersomatic cage 1 is intended to be inserted and set in place inside the body of the patient laterally, that is to say by the side of the patient, its shape and the arrangement of its components being adapted to such an insertion mode. A lateral insertion of the cage 1, advantageously according to a direction of insertion substantially orthogonal to the extension plane of the spinous processes 4A, 5A, which substantially corresponds to the anatomical sagittal plane, as is particularly visible in FIGS. 15 and 16, advantageously allows positioning said cage 1 between the first vertebral body 4 and the second vertebral body 5 by performing a relatively small-sized incision in the tissues of said patient, while avoiding interferences with the nerves and the vessels affixed to said vertebral bodies. The approach is located at the level of the psoas. Preferably, the set-up of the cage in the body of the patient is performed using a dilator tube and/or a distractor, in minimally invasive surgery. Of course, other methods for setting the cage 1 in place may be considered, the shape and the design of said cage 1 being adapted accordingly without departing from the scope of the invention. In particular, the cage 1 may be introduced in the body of the patient by an anterior, or posterior, first approach.

As is particularly visible in FIG. 3 representing an exploded view of an example of a cage 1 in accordance with the invention, as well as in FIGS. 1 and 2, the cage 1 of the invention comprises three main portions described hereinafter, namely a cage body 2, an expansion or adjustable cap 3 pivotally mounted on the cage body 2, and a means 16 for controlling the inclination of the expansion cap 3 with respect to the cage body 2.

According to the invention, the cage 1 first comprises a cage body 2, comprising a bearing surface 6A intended to be positioned so as to bear against the first vertebral body 4. Advantageously, the cage body 2 is generally parallelepiped shaped, and preferably forms most of the bulk of the cage 1. Of course, other shapes may be considered depending on the location in which said cage 1 might be implanted, in order to adapt to the morphology of the patient.

According to the invention, the cage body 2 constitutes the element of the cage 1 by which said cage 1 is intended to rest on the first vertebral body 4, and in particular against the vertebral endplate of the latter. Preferably, the cage body 2 forms a lower portion of the cage 1, the first vertebral body 4 being placed below the second vertebral body 5, as illustrated in FIGS. 1, 2, 15 and 16. Nonetheless, the reverse arrangement may be adopted, the first vertebral body 4 may be placed, on the contrary, above the second vertebral body 5 (which is not illustrated). In order to simplify the description, it will, nonetheless, be considered that in the case of the illustrated example, the cage body 2 is placed so as to form the lower portion of the cage 1.

The cage body 1 is intended to rest, and to bear, on the first vertebral body 4 via the bearing surface 6A, which includes for this purpose a shape adapted to the morphology of said first vertebral body 4, preferably substantially planar. As in the case of the example illustrated in the figures, the cage body 2 preferably comprises an external envelope 7 having a generally parallelepiped shape, and enclosing a hollow space E. Preferably, the external envelope 7 forms a hollow pocket with a generally parallelepiped shape, so that the hollow space E advantageously opens onto a gaping access opening 8, bordered by four tapering and vertical walls 9, 10, 11 disposed substantially as a rectangle, being for example parallel in pairs, and contributing to form the external envelope 7.

As illustrated in the figures, the external envelope 7 preferably forms a bottom plate 6 forming said bearing surface 6A, which is preferably disposed opposite the access opening 8, at the bottom of the hollow pocket of the external envelope 7, so as to link the four walls 9, 10, 11 to each other. Thus, the cage body 2 preferably rests on the first vertebral body 4 via the bottom plate 6, as illustrated in particular in FIGS. 1 and 2. Preferably, such a design allows the hollow external envelope 7 and the cage body 2 to have a structure which is sufficiently rigid to resist the compression force exerted by the first vertebral body 4 and the second vertebral body 5 against the cage 1, while allowing the latter to be relatively light.

According to a major feature of the invention, said cage body 2 comprises at least one fastening orifice 41 forming an oblique well 42 capable of receiving and guiding from the outside of said cage 1 a means 43 for fastening the cage 1 to the first vertebral body 4 and/or to the second vertebral body 5.

Preferably, this fastening means 43 comprises a rigid and elongated body, such as for example, a wire, a blade, a nail or a screw, as illustrated as example in the figures.

In order to enable the passage of such a fastening means 43, said cage body 2 includes, as stated hereinabove, a bottom plate 6, the latter being provided with at least one primary through hole 44 through which said fastening means 43 introduced by the fastening orifice 41 can open so as to fasten, or at least contribute to fasten, said cage body 2 to the first vertebral body 4.

As illustrated in particular in FIGS. 5 to 14, said oblique well 42 and primary through hole 44 are advantageously designed, orientated and positioned with respect to each other, so that said fastening means 43 being inserted from the outside of the cage 1 in said oblique well 42 of the fastening orifice 41, the primary through hole 44 is located on the natural path, that is to say that induced by the shape and the inclination of the internal wall of said oblique well 42, of said fastening means 43. The latter, preferably a wing screw type, will be advantageously chosen with a sufficient length so that, its head 46 being housed in the oblique well 42, a portion of its length opens from the primary through hole 44 arranged in the bottom plate 6 of the cage body 2 so as to be anchored by screwing in the first vertebral body 4, for example in the cortical bone or in the osseous tissue of this vertebral body 4. Moreover, said fastening orifices 41 and/or fastening means 43 may advantageously be shaped or designed to be self-retaining in order to guarantee the durability of the holding of the cage 1 in the intersomatic space. Thus, such a fastening of the cage body 2 contributes to guarantee the proper stability of the cage 1 between the vertebrae.

Furthermore, the fastening orifice 41 is optionally adapted to enable the connection of a means for introducing and delivering an osteoinductive material, through said fastening orifice 41, inside the cage 1. Accordingly, the surgeon can, at his discretion, make use successively of said fastening orifice 41 in order to introduce the osteoinductive material in the cage 1 and then guide a fastening means 43, or use the fastening orifice 41 only for the introduction of the osteoinductive material, without implementing any fastening means 43 or, conversely, use it only to guide a fastening means 43.

FIG. 3 represents a first example of an expansion cap 3 in accordance with the invention, and belonging to the cage 1 of FIGS. 1, 2 and 4. FIGS. 9 to 14 show a second embodiment thereof, in connection with the cage 1 of FIGS. 5 to 8. As illustrated in these figures, the extension cap 3 is preferably in the form of a one single piece. However, it may perfectly comprise several distinct extension cap 3 elements.

According to the invention, the expansion cap 3 is mounted on the cage body 2 via a linking element 12, 13, so as to be able to pivot according to an inclination stroke about an axis of inclination X-X′ relative to the bearing surface 6A. Thus, the expansion cap 3 is preferably rotatably mounted on the cage body 2 opposite to the bearing surface 6A and to the bottom plate 6, in a swinging manner about the axis of inclination X-X′, in particular so as to close the access opening 8 in the same manner as a swinging door. To this end, the linking element 12, 13 advantageously forms a pivot linkage about the axis of inclination X-X′, linking the expansion cap to the cage body 2. In turn, the expansion cap 3 preferably forms the upper portion of the cage 1, which may be orientated about the axis of inclination X-X′ in a continuous manner. Preferably, the expansion cap 3 forms a cap plate with a generally substantially rectangular shape, and delimited by a cap edge 40.

In particular, the cap edge 40 comprises, on the one hand, a first edge connected to the cage body 2 via the linking element 12, 13 so as to be able to pivot, preferably freely, and a swinging free edge opposite to said connected edge. Thus, the linking element 12, 13 preferably extends along said cap edge 40, but may alternatively extend in a median portion of the cap (not illustrated) without departing from the scope of the invention. The cage body 2, and in particular the external envelope 7, advantageously comprises a longitudinal support wall 9, that is to say extending in the longitudinal direction of the external envelope 7 with a generally parallelepiped shape, and which advantageously forms one of the aforementioned walls 9, 10, 11, said longitudinal support wall 9 protruding from the bottom plate 6 up to a support edge 14, the expansion cap 3 being mounted on said support wall 9 of the cage body 2 via the linking element 12, 13, in the vicinity of the support edge 14. According to such a design, the support wall 9 preferably forms a substantially planar wall, for example substantially perpendicular to the bottom plate 6, and advantageously extending from the bottom plate 6 up to the axis of inclination X-X′, along which extend the support edge 14 and the linking element 12, 13. Preferably, the external envelope 7 of the cage body 2 forms a front wall 10 protruding from the bottom plate 6 up to a stop edge 15. The front wall 10 is then preferably disposed opposite the support wall 9, for example parallel to the latter so that the support wall 9, the front wall 10 and the bottom plate 6 form a <<U >>, the hollow space E being arranged between said walls 9, 10.

The expansion cap 3 of the invention comprises an element 3A for bearing against the second vertebral body 5, that is to say that the second vertebral body 5 is intended to rest on said cap 3 via the bearing element 3A. As illustrated in the figures, the bearing element 3A is formed for example by an upper surface 3A of the cap plate forming the expansion cap 3. Of course, the bearing element 3A may nonetheless be formed for example by an edge of the expansion cap 3, or by a non-illustrated additional part attached on the cap 3 without departing from the scope of the invention. Preferably, the external envelope 7 also forms at least a first transverse wall 11 linking the front wall 10 to the support wall 9, and protruding from the bottom plate 6. The external envelope 7 may also comprise a second transverse wall 11 disposed opposite the first transverse wall 11, for example parallel to the latter. Preferably, the hollow space E is formed between the first transverse wall 11, the front wall 10, the support wall 9 and the bottom plate 6, optionally as well as the second transverse wall 11.

FIGS. 1-2, 9-10, 11-12 and 13-14 represent examples of different inclinations which can be adopted by the expansion cap 3. Thus, depending on the inclination of the expansion cap 3, the cage 3 is more or less extended, which allows in particular influencing on the relative positioning, and/or on the relative orientation, of the first vertebral body 4 with respect to the second vertebral body 5 which are bearing against each other via said cage 1. Preferably, the inclination stroke of the expansion cap 3 relative to the bearing surface 6A extends between:
on the one hand, a minimum inclination of said expansion cap 3 (as illustrated for example in FIG. 1) in which the bearing element 3A, and in particular the expansion cap 3, is at a minimum distance from the bearing surface 6A, and
on the other hand, a maximum inclination of said expansion cap 3 (as illustrated for example in FIG. 2) in which the bearing element 3A is at a maximum distance from the bearing surface 6A.

In its minimum inclination, the expansion cap 3, and in particular its free edge, is the closest to the cage body 2, and in particular to the bearing surface 6A. Thus, the space occupied by the cage 1 in this situation is minimum, so that said cage 1 is in a compact configuration allowing it for example to be easily introduced in the body of the patient, and in particular in the targeted intersomatic space. The minimum inclination of the expansion cap 3 corresponds to a situation in which said expansion cap, and/or the bearing element 3A, extends in a plane substantially parallel to the bearing surface 6A, and/or to the bottom plate 6, as is the case in the example of FIG. 1. Of course, the minimum inclination may correspond to a situation in which said expansion cap, and/or the bearing element 3A, are not parallel to the bearing surface 6A, and/or to the bottom plate 6. When the cage 1 is inserted between the first vertebral body 4 and the second vertebral body 5 with its expansion cap 3 in the minimum inclination, the correction of the relative position of said vertebral bodies brought by said cage 1 is also minimal. Advantageously, the expansion cap 3 abuts against the stop edge 15 of the front wall 10 when it is in its minimum inclination so that the inclination stroke of the expansion cap 3 is limited by the stop edge 15. Advantageously, the stop edge 15 allows forming an end-of-stroke of the inclination of the expansion cap 3. Thus, the expansion cap 3 is preferably brought, in this situation, to close and cover all or part of the access opening 8. In its minimum inclination, the expansion cap 3 is preferably folded over, or even retracted, against the cage body 2. Alternatively, the inclination end-of-stroke of the expansion cap 3 may, of course, be formed intrinsically in the linking element 12, 13, or by other walls 9, 10, 11, or other elements of the cage body 2.

The expansion cap 3 may also be open, preferably substantially freely, up to its maximum inclination, so that said expansion cap 3, and/or the bearing element 3A can be inclined according to an angle of inclination a comprised between about 0° (minimum inclination) and 30° (maximum inclination) with respect to the bearing surface 6A or the bottom plate 6, about the axis of inclination X-X′. Thus, in its maximum inclination, the expansion cap 3 is at a maximum distance from the bearing surface 6A and/or from the bottom plate 6, so that the cage 1 is in an extended configuration, and has a maximum bulk. In this maximum inclination, the orientation and/or the positioning of the first vertebral body 4 with respect to the second vertebral body 5 is modified in a relatively considerable manner, which allows causing, creating, or on the contrary attenuating, a lordosis, a kyphosis, or a scoliosis of the spine of the patient. Indeed, advantageously, said vertebral bodies 4, 5, and in particular their respective vertebral endplates, rest on the bearing surface 6A and on the bearing element 3A, so that they adopt the inclination imposed by the cage 1, and in particular that of its expansion cap 3.

Preferably, said expansion cap 3 is, like the previously described bottom plate 6, provided with at least one secondary through hole 45 through which a fastening means 43 introduced by a fastening orifice 41 that the cage body 2 comprises can open so as to fasten, or at least contribute to fasten, said cage body 2 to the second vertebral body 5. Advantageously, this secondary through hole 45 is configured so as to enable the passage of said fastening means 43 regardless of the angle of inclination of the expansion cap 3 with respect to the bearing surface 6A.

In particular, said oblique well 42 and secondary through hole 45 are advantageously designed, orientated and positioned relative to each other, so that said fastening means 43 being inserted from the outside of the cage 1 in said oblique weld 42 of the fastening orifice 41, the secondary through hole 45 is located on the natural path, that is to say that induced by the shape and the inclination of the internal wall of said oblique well 42, of said fastening means 43. Furthermore, said secondary through hole 45, for example with a circular or oblong shape, is shaped and sized so that the fastening means 43 could pass in the secondary through hole 45 without interfering with the expansion cap 3, and regardless of the inclination of the latter.

As illustrated as example in FIGS. 9 to 14, the secondary through hole 45 is advantageously oblong and extends longitudinally over a length L, parallel to the axis of inclination X-X′, and transversely over a width I. Advantageously, these dimensions I and L are chosen in accordance with the foregoing, so that the secondary through hole 45 is maintained on the path of said fastening means 43 and the latter could be implemented through this secondary through hole 45 regardless of the angle of inclination chosen by the surgeon of the expansion cap 3 with respect to the bearing surface 6A.

In a particularly advantageous manner and as illustrated as example in FIGS. 5 to 16, said cage body 2 comprises at least two fastening orifices 41 forming respectively an oblique well 42, one of said fastening orifices 41 being intended to receive and guide from the outside of said cage 1 a means 43, for example a screw, for fastening the cage 1 to the first vertebral body 4 via the primary through hole 44 that the bottom plate 6 is provided with, and the other fastening orifice 41 being in turn intended to receive and guide from the outside of said cage 1 a means 43 for fastening the cage 1 to the second vertebral body 5 via the secondary through hole 45 that the expansion cap 3 is provided with. Thus, such a fastening of the cage body 2, using at least two fastening means 43 passing obliquely throughout the cage 1, contributes to guarantee an excellent stability of the cage 1 between the vertebrae, regardless of the compression, but also shear, forces exerted by the vertebral bodies 4, 5.

In order to obtain the imposed inclination effect of the expansion cap 3 described hereinbefore, it is necessary to provide for a means for maintaining the latter in position. Consequently, in order to position and maintain said expansion cap 3 in a given inclination of its inclination stroke, the intersomatic cage 1 also comprises a means 16 for controlling the inclination of the expansion cap 3 capable of maintaining the expansion cap 3 in a desired inclination of the inclination stroke, an embodiment of which is particularly visible in FIG. 4, in an exploded view. The control means 16 simultaneously allows:
the surgeon to control the inclination of the expansion cap 3 via said control means 16, in particular when the cage 1 is already implanted in the body of the patient, preferably when said cage 1 is positioned in the intersomatic space bearing against at least one of the vertebral bodies 4, 5, and
maintaining said expansion cap 3 in its inclination, and this in spite of the possible opposing compression forces generated by the first vertebral body 4 and the second vertebral body 5.

Preferably, the control means 16 is accessible to the surgeon when the cage 1 is in place in the intersomatic space, so that the latter could set the inclination of the expansion cap 3 after the set-up. For example, as is visible in FIGS. 1 and 2, the control means 16 comprises a head for interacting with a tool 47, such as a screw head 18, which appears in an orifice arranged in the first transverse wall 11 of the cage 1, said transverse wall 11 facing the surgeon when said cage 1 is in place in the body of the patient.

Ultimately, when the cage 1 is implanted between the first vertebral body 4 and the second vertebral body 5, it therefore undergoes from the latter opposing compression forces, which are received by upper and lower ends of said cage 1, formed in this case by the bearing surface 6A and by the bearing element 3A, and transmitted to the walls 9, 10, 11, and to the control means 16, which absorb said opposing compression forces so as to maintain said vertebral bodies 4 and 5 in position. The control means 16 allowing performing a setting of the inclination of the expansion cap 3, the cage 1 allows a set-up of the cage in an accurate, reliable, and modifiable way so as to treat a pathology in a satisfactory manner. Indeed, preferably, the cage 1 is introduced in the body of the patient by the surgeon in the minimum inclination, and set in place in the intersomatic space in this configuration. Afterwards, the surgeon can then advantageously make the inclination of the expansion cap 3 vary via the control means 16 in order to apply to the spine of the patient the desired angular correction, or the desired position correction.

The description will henceforth cover in more detail the control means 16, which may, as such, be the object of a protection on its own.

Advantageously, the control means 16 comprises at least a first screw 17 for controlling the inclination of the expansion cap 3 operable by a surgeon, and having an own screwing axis Y-Y′. Advantageously, the first control screw 17 is provided with a screw head 18 by which said screw 17 can be rotated about its screwing axis Y-Y′ by the surgeon from the outside of the cage 1.

The control means 16 may be made according to two main variants, one in which the screw head 18 is preferably accessible from one of the walls 9, 10, 11 of the cage body 2 (as illustrated for example in FIGS. 1 and 2), and the other in which the screw head 18 is accessible from the edge of the cap 3 (not represented). Consequently, the first control screw 17 is preferably rotatably mounted about its screwing axis Y-Y′ either on the cage body 2, or on the expansion cap 3. For example, the first control screw 17 is mounted in free rotation on the cage body 2 via rotation orifices 19 passing through the first transverse wall 11 and/or the second transverse wall 11, so that at least one of said transverse walls 11 supports the first control screw 17. Advantageously, the control screw 17 comprises one or several non-threaded portion(s) by which it is mounted in the rotation orifices 19, so that said first control screw 17 does not advance along its screwing axis Y-Y′ relative to the cage body 2 (or respectively relative to the cap 3) when it is rotated about said screwing axis Y-Y′. Besides the rotation orifice 19, the cage body 2 or the expansion cap 3 may comprise an intermediate cradle 23 for rotatably supporting the first control screw 17, allowing maintaining said control screw 17 on an intermediate or central portion of the latter, while enabling its rotation.

Preferably, the first control screw 17 comprises a first thread 20 about the screwing axis Y-Y′ so that the rotation of the screw 17 about its axis Y-Y′ causes an advance movement of the first thread 20 either relative to the cage body 2 if said first control screw 17 is mounted on the latter, or relative to the expansion cap 3 if said first control screw 17 is mounted on the latter.

Preferably, the control means 16 also comprises a first transmission means allowing transforming the advance movement of the first thread 20 into an inclination movement of the expansion cap 3 relative to the cage body 2. In this advantageous situation, advantage is taken from the presence of the first thread 20 which serves both to control the inclination of the expansion cap 3 via the first control screw 17 and to maintain the latter, the thread having for example a pitch which is sufficiently large to substantially prevent any reversibility of the mechanism, and in particular a rotation of the first control screw 17 via the transmission means. Thus, the cage 1, and its orientation setting are particularly reliable, and easy to handle.

As represented as example in the exploded view of FIG. 4, preferably, the first transmission means comprises at least a first mobile 24 which comprises a threaded orifice 26 by which it is mounted on the first control screw 1. Thus, the first thread 20 preferably cooperates with the threaded orifice 26 so as to transmit its advance movement to the first mobile 24 along the screwing axis Y-Y′. Advantageously, the first mobile 24 evolves within the hollow space E.

Preferably, the first transmission means also comprises a first means for blocking in rotation about the screwing axis Y-Y′ the first mobile 24, so that the latter translates along the screwing axis Y-Y′ under the action of the rotation of the first control screw 17 about the latter. In the case where the first control screw 17 is mounted on the cage body 2, the blocking means will allow blocking the rotation about the screwing axis Y-Y′ of the first mobile 24 relative to said cage body 2. Advantageously, the blocking means is formed by a guide rail internal to the hollow space E, formed for example by the front wall 10, and/or a central rib 27, and/or the bottom plate 6 (in the case represented in FIG. 4, as well as in FIG. 8). Of course, another one of the walls 9, 10, 11 may also or alternatively contribute to form the guide rail. In the alternative case where the first control screw 17 is mounted on the expansion cap 3, the blocking means will allow blocking the rotation about the screwing axis Y-Y′ of the first mobile 24 relative to said expansion cap 3. In this second preferred case, the blocking means will be for example formed by an aperture 30 of the cap the edges of which form a rail for guiding and blocking the rotation of the first mobile 24.

Regardless of its rotation blocking means, the first mobile 24 is preferably in contact respectively:
either with the expansion cap 3 in the preferred case where the first control screw 17 is mounted on the cage body 2,
or with the cage body 2, in the preferred case where the first control screw 17 is mounted on the expansion cap 3,

so that its translational movement causes a variation of the inclination of said expansion cap 3.

In this case, the rotation of the first control screw 17 advantageously causes the displacement of the first mobile 24 according to the advance movement of the first thread 20, so that said first mobile 24 is brought:
either to push the expansion cap 3 in the preferred case where the first control screw 17 is mounted on the cage body 2,
or to push the cage body 2 in the preferred case where the first control screw 17 is mounted on the expansion cap 3.

The first mobile 24 being mounted on the first thread 20, it cannot advantageously be displaced by compression of the expansion cap 3 against the cage body 2, so that the control means 16 maintains the expansion cap 3 in its inclination against the compression forces exerted by the vertebral bodies 4, 5.

Preferably, in order to allow a particularly accurate setting of the inclination of the expansion cap 3, the first mobile 24 comprises a first deflection surface 31, for example inclined with respect to the screwing axis Y-Y′, the first transmission means comprising at least a first deflection element 28, for example inclined with respect to the axis of inclination X-X′ at an angle close to or mutual with that of the deflection surface 31. Preferably, the first deflection element 28 is secured respectively:
either to the expansion cap 3, in the preferred case where the first control screw 17 is mounted on the cage body 2,
or to the cage body 2, in the preferred case where the first control screw 17 is mounted on the expansion cap 3.

Preferably, via the first deflection element 28, the first control screw 17 causes the inclination of the expansion cap 3, the first deflection element 28 and the first deflection surface 31 being in sliding contact on each other, so that said first deflection element 28 is driven by the first deflection surface 31, during the translation of the first mobile 24, in a displacement having at least one non-zero displacement component according to an axis Z-Z′ orientated in a circular manner, that is to say orthoradial, with respect to the screwing axis Y-Y′. In other words, the complementary and reciprocal shapes of the first deflection element 28 and of the deflection surface 31 advantageously allow converting the orientation of the advance movement of the first mobile 24 into an opening movement of the expansion cap 3, that is to say the increase of its inclination.

Preferably, the first deflection element 28 has a zero displacement component according to the screwing axis Y-Y′, so that substantially all the advance movement of the first thread 20 is advantageously deflected radially with respect to the axis Y-Y′ so as to allow opening of the expansion cap 3 with a maximum mechanical efficiency. The shape of the first deflection element 28 and of the first deflection surface 31 will be adapted accordingly, in particular according to the arrangement of the axis of inclination X-X′ with respect to the screwing axis Y-Y′.

Preferably, as illustrated in FIG. 4 in an exploded view, and in FIGS. 1 and 2, the first control screw 17 is rotatably mounted on the transverse wall 11 of the cage body 2. Preferably, said first control screw 17, regardless of its mounting location, is mounted so that the screwing axis Y-Y′ is substantially parallel to the axis of inclination X-X′ of the expansion cap 3, which allows access to the screw head 18 by the side of the cage 1, advantageously through one of the rotation orifices 19, so that the latter is accessible by the surgeon during a first lateral insertion. Thus, the cage 1 is particularly easy to implant and to set.

In this preferred configuration, said at least one fastening orifice 41 is, as illustrated in the figures, preferably arranged in said transverse wall 11 of the cage body 2, so as to be also accessible by the surgeon during a first lateral insertion so that the latter could insert therein a means 43 for fastening the cage 1 to the first vertebral body 4 and/or to the second vertebral body 5.

Preferably, in order to improve the accuracy, the reliability and the mechanical strength of the control means 16, the first control screw 17 comprises a second thread 21 about the screwing axis Y-Y′, the pitch of which is preferably reversed with respect to the first thread 20, the rotation of the first control screw 17 about its screwing axis Y-Y′ resulting in an advance movement of the second thread 21. Optionally, this advance movement may thus be performed, depending on the direction of the pitch, in a direction opposite to that of the first thread 20.

In this preferred case, the control means 16 comprises a second transmission means allowing transforming the advance movement of the second thread 21 into an inclination movement of the expansion cap 3 relative to the cage body 2, via a second mobile 25, so that the first thread 20 and the second thread 21 contribute together to the control of the inclination of the expansion cap 3 during the rotation of the first control screw 17.

Preferably, the action of the second mobile 25 on the inclination of the expansion cap 3 is similar to that of the first mobile 24, and is advantageously mutual in the advantageous case where the pitch of the second thread 21 is reversed with respect to that of the first thread 20. Consequently, a second deflection element 29 and a second deflection surface 32 are provided, with a design similar to the first deflection element 28 and to the first deflection surface 31 respectively.

Preferably, the first deflection element 28 and the second deflection element 29 are disposed head-to-tail on an internal face 33 of the expansion cap 3, so as to protrude from the latter, said internal face 33 being turned to the side of the hollow space E. Similarly, the first deflection surface 31 and the second deflection surface 32 are disposed head-to-tail along the first control screw 17. Advantageously, this head-to-tail mounting allows cancelling the forces in the direction of the screwing axis Y-Y′ communicated by the mobiles 24, 25 to the expansion cap 3 (or respectively to the cage body 2).

A second control screw may be provided, preferably rotatably mounted via rotation orifices 19 of the cage body 2, and disposed for example so that its screwing axis is parallel to the screwing axis Y-Y′ of the first control screw 17. Advantageously, a similar or different second transmission means similar will then be provided to transform the rotation of the second screw into an inclination of the expansion cap 3.

The description will now cover in more detail the linking element 12, 13, which may also, as such, be the object of a protection on its own. The details of an embodiment of a linking element 12, 13 in accordance with the invention appear in FIGS. 1 to 14.

Preferably, the cage 1 comprises a hinge forming the linking element 12, 13, and by which the expansion cap 3 is pivotally mounted about the axis of inclination X-X′ on the cage body 2. Advantageously, the hinge is formed, on the one hand, by a first hinge element 12, which is optionally integral with the cage body 2 and, on the other hand, by a second hinge element 13, which is optionally integral with the expansion cap 3. In this advantageous case, the first hinge element 12 and the second hinge element 13 are designed to cooperate with each other in order to form the hinge, and in particular to be rotative relative to each other about the axis of inclination X-X′, while ensuring securing of the expansion cap 3 and of the cage body 2. In this configuration, the first hinge element 12 and the second hinge element 13 form alone said hinge, without the necessity to implement an element, such as for example a knuckle pin, for blocking said first and second hinge elements 12, 13. Nonetheless, alternatively, the linking element 12, 13 may be formed by an independent part, such as a metallic or plastic axis, which is an attached part and which ensures the linkage and pivoting of the expansion cap 3 with the cage body 2.

Preferably, the first hinge element 12 comprises a rectilinear groove 34 arranged in the cage body 2 and extending along the axis of inclination X-X′, the rectilinear groove 34 comprising a retaining flange 35 extending along the axis of inclination X-X′ over at least part of the length of the rectilinear groove 34, preferably over its entire length. Thus, the rectilinear groove 34 and its retaining flange 35 form an internal chamber preferably with a cylindrical shape, along which is arranged a longitudinal opening delimited by the retaining flange 35, the longitudinal opening being finer than the diameter of said cylindrical chamber. Preferably, the longitudinal opening, optionally as well as the retaining flange 35, extends substantially over the entire length of said rectilinear groove 34, or at least over most of the latter.

Advantageously, the second hinge element 13 comprises a rotation rod 36 having an own longitudinal axis, which is preferably substantially cylindrical over at least most of its circumference.

As is visible in particular in FIGS. 1 and 2, the rotation rod 36 is nested in the rectilinear groove 34 so that said own longitudinal axis of the rotation rod 36 is substantially coaxial with the axis of inclination X-X′. To this end, the rectilinear groove 34 is preferably shaped so that the rotation rod 36 could perform a rotation about said axis of inclination X-X′ relative to the first hinge element 12, the retaining flange 35 allowing retaining the rotation rod 36 within the rectilinear groove 34. Thus, the respective shapes of the rotation rod 36 and of the rectilinear groove 34 allow the rod to be trapped in the rectilinear groove 34 while being rotative about the axis of inclination X-X′, which advantageously allows avoiding any translation of the expansion cap 3 relative to the cage body 2 according to the axis of inclination X-X′, for example, under the effect of vibrations or mechanical stresses relating to the compression force exerted on the cage 1 by the first and second vertebral bodies 4, 5.

Preferably, the second hinge element 13 comprises a tab 37 for fastening the rotation rod 36 to the expansion cap 3, said fastening tab 37 extending from the expansion cap 3 up to the rotation rod 36 over at least most of the length of the rotation rod 36. Thus, the fastening tab 37 is designed to secure and maintain the rotation rod 36 at a distance from the expansion cap 3. Preferably, the fastening tab 37 links the edge of the expansion cap 3 to the rotation rod 36. Advantageously, the upper surface 3A of the cap is prolonged by the fastening tab 37 as is particularly visible in FIG. 3. Thus, the upper surface 3A of the cage 1 is advantageously devoid of any protruding ridge and therefore adapts particularly well to the shape of the second vertebral body 5.

Preferably, the fastening tab 37 has a thickness smaller than the width of the longitudinal opening of the rectilinear groove 34. Preferably, the fastening tab 37 circulates from one edge to another of the longitudinal opening of the rectilinear groove 34 according to the inclination adopted by the expansion cap 3 with respect to the cage body 2. Preferably, the rectilinear groove 34 as well as the fastening tab 37 extend substantially over the entire length of the hinge, from one end to another of the latter. Possibly, the fastening tab 37 may abut against the retaining flange 35 in order to define the maximum and/or minimum inclination of the expansion cap 3.

Furthermore, the expansion cap 3 preferably comprises at least one aperture 30, or more, arranged in the latter so as to pass therethrough, so as to enable the introduction of an osteoinductive material (for example a bone graft) through said aperture 30, inside the cage 1, and in particular in the hollow space E. As illustrated in FIGS. 3 and 4, the aperture(s) 30 may have for example an oblong shape. As previously described, such apertures 30 may also advantageously participate in forming the means 16 for controlling the opening of the expansion cap 3, serving in particular as a means for guiding in translation mobiles 24, 25 placed inside the hollow space E.

In all cases, the mobiles 24, 25 may advantageously allow compressing or compacting the osteoinductive material within the hollow space E.

As illustrated in FIGS. 9 to 14, said aperture 30, or one of these apertures 30, may advantageously be coincident with the secondary through hole 45 through which said fastening means 43 introduced by the fastening orifice 41 can open so as to fasten said expansion cap 3 to the second vertebral body 5. Alternatively, it may, of course be completely distinct from this secondary through hole 45 without departing from the scope of the invention.

Preferably, the bottom plate 6 and/or either one of the walls 9, 10, 11 comprise a plurality of pores 48 which pass therethrough, the passage section of said pores having a characteristic variable comprised between 0.01 mm and 5 mm. For example, the characteristic variable of the passage section forms its diameter, its smallest width, or its largest width. Advantageously, such pores 48 may be made by an additive manufacturing process, for example a three-dimensional printing, or a laser melting, and preferably measure between 0.05 and 0.03 mm. Alternatively, conventional machining means may be used for making said pores 48, which will be therefore larger, and will measure for example between 0.5 and 5 mm. Advantageously, the pores may form through channels, for example in a honeycomb fashion.

Alternatively, said bottom plate 6 and/or the support wall 9 and/or the front wall 10 and/or the transverse wall 11 may also be provided, in addition to said pores 48 and for the same purposes, with through apertures 30 (FIGS. 5 to 7, 9 and 10) or be provided only with these said apertures 30, with the exclusion of said pores 48 (FIGS. 8, 13 and 14).

Preferably, the bottom plate 6 comprises an inner surface 38 opposite to the bearing surface 6A, an osteoinductive material being intended to be attached in the hollow space E on said inner surface 38, so that a bone fusion could take place between the first vertebral body 4 and the osteoinductive material via the pores 48 of said bottom plate 6. Thus, the cage body 2 may be fastened to the first vertebral body 4 via the osteoinductive material, which is less invasive than fastening by screwing for example. In the same manner, pores 48 may be arranged in the expansion cap 3 in order that it could be fastened in the same manner to the second vertebral body 5 via an osteoinductive material.

Advantageously, the upper surface 3A and the bearing surface 6A may be rough so as to enable a retention of the cage 1 by rough adherence, in particular under the effect of the compression forces of the vertebral bodies 4, 5 on the cage 1.

Preferably, each of the cage body 2 and the expansion cap 3 are made into one single piece, for example by machining blank parts, or by additive manufacturing processes. Nonetheless, as stated hereinbefore, the expansion cap 3 and, optionally, the cage body 2 may alternatively be made each into several parts.

In order to set the cage 1 of the invention in place, it is possible for example to implement a surgical method including the following steps:
performing an opening of the body of the patient using a lateral approach leading to the intersomatic space between the desired first vertebral body 4 and second vertebral body 5,
preparing the intersomatic space to accommodate the cage 1,
the expansion cap 3 being in the minimum inclination, introducing an osteoinductive material in the hollow space of the cage 1 via the apertures 30 and/or the fastening orifice 41,
introducing the cage 1 in the intersomatic space, the expansion cap 3 of the latter being in its minimum inclination, preferably laterally, and still more preferably so that the axis of inclination X-X′ of the expansion cap 3 with respect to the bearing surface 6A is orthogonal to the plane of extension of the spinous processes 4A, 5A of the first and second vertebral bodies 4, 5, which plane substantially corresponds to the anatomical sagittal plane,
increasing and setting the inclination of the cage 1 via the control means 16, for example using a tool 47 for interacting with the screw head 18 of the latter, so as to obtain the desired correction for the patient, as illustrated for example in FIG. 15,
fastening the cage 1 using at least one fastening means 43 inserted from the outside of said cage 1 in a fastening orifice 40, so as to fasten the cage 1 to the first vertebral body 4 and/or to the second vertebral body 5, as illustrated for example in FIG. 16,
closing the body of the patient.

In order to remove the cage 1 from the body of the patient, the surgeon performs for example the previous steps in the reverse order.

Moreover, the invention concerns, as such, a method for manufacturing an expandable intersomatic cage 1 intended to be implanted between a first vertebral body 4 and a second vertebral body 5 of a patient, said cage 1 preferably being in accordance with the previous description.

The method of the invention includes a step during which a cage body 2 is made, comprising a bearing surface 6A intended to be positioned so as to bear against the first vertebral body 4, and a step during which an expansion cap 3 is made comprising an element 3A for bearing against the second vertebral body 5.

Moreover, the method of the invention includes a step during which the expansion cap 3 is mounted on the cage body 2 via a linking element 12, 13, so that the expansion cap 3 could pivot according to an inclination stroke about an axis of inclination X-X′ relative to the bearing surface 6A, the inclination stroke preferably extending between:
on the one hand, a minimum inclination of said expansion cap 3 in which the bearing element 3A is at a minimum distance from the bearing surface 6A, and
on the other hand, a maximum inclination of said expansion cap 3 in which the bearing element 3A is at a maximum distance from the bearing surface 6A.

Preferably, the linking element 12, 13 is in accordance with the previously described one. Thus, the method advantageously includes, preferably prior to the assembly of the expansion cap 3 on the cage body 2, the following steps:
making, on the one hand, a first hinge element 12 integral with the cage body 2, the first hinge element 12 advantageously comprising a rectilinear groove 34 arranged in the cage body 2 and extending along the axis of inclination X-X′, the rectilinear groove 34 extending between two groove ends 39 at least one of which is open.
making, on the other hand, a second hinge element 13 integral with the expansion cap 3, the second hinge element 13 advantageously comprising a rotation rod 36 having an own longitudinal axis.

Thus, the step of assembling the expansion cap 3 with the cage body 2 may preferably be performed by assembling the first hinge element 12 with the second hinge element 13 in order to form a hinge forming the linking element 12, 13. For this purpose, the second hinge element 13 is preferably nested on the first hinge element 12 by fitting the rotation rod 36 in the rectilinear groove 34 via the open groove end 39 and by making said rotation rod 36 slide along said rectilinear groove 34 along the axis of inclination X-X′, up to the other groove end 39, so that the entire length of the rotation rod 36 is advantageously comprised between the two groove ends 39. Preferably, the rectilinear groove 34 and the rotation rod 36 are shaped so that their sliding in each other has to be performed forcibly.

According to the invention, a means 16 for controlling the inclination of the expansion cap 3 capable of maintaining the expansion cap 3 in a desired inclination of the inclination stroke, is made, or provided. Preferably, the control means 16 is in accordance with the previous description. Preferably, the control means 16 is assembled to the expansion cap 3 and/or to the cage body 2 prior to the step of assembling said expansion cap with the cage body 2.

Still according to the invention, said manufacturing method also comprises a step during which said cage body 2 is provided, for example by drilling after making of the cage body 2 or by matter omission when making the cage body 2, with at least one fastening orifice 40 forming an oblique well 41 capable of receiving and guiding from the outside of said cage 1 a means 43 for fastening the cage 1 to the first vertebral body 4 and/or to the second vertebral body 5. Preferably, this fastening orifice 41 is also in accordance with the previous description.

Preferably, at least the step of making the cage body 2 is performed using an additive manufacturing process, preferably a laser melting process. A manufacture by conventional machining may also be considered.

The cage 1, and in particular the expansion cap 3 and the cage body 2, may be made of a biocompatible material such as PEEK, PEKK, or of titanium. Besides, these elements may preferably be made either of SLS, or of laser melting titanium if this manufacturing mode is retained.

### POSSIBILITY OF INDUSTRIAL APPLICATION

The invention finds its industrial application in the design, the manufacture and the implementation of an expandable intersomatic cage intended to be implanted between a first vertebral body and a second vertebral body of a patient.

1. An expandable intersomatic cage (1) intended to be implanted between a first vertebral body (4) and a second vertebral body (5) of a patient, said cage (1) comprising:
a cage body (2), comprising a bearing surface (6A) intended to be positioned so as to bear against the first vertebral body (4),
an expansion cap (3), comprising an element (3A) for bearing against the second vertebral body (5), and mounted on the cage body (2) via a linking element (12, 13) so as to be able to pivot according to an inclination stroke about an axis of inclination (X-X′) relative to the bearing surface (6A),
a means (16) for controlling the inclination of the expansion cap (3) capable of maintaining the expansion cap (3) in a desired inclination of the inclination stroke,
said cage (1) being characterized in that said cage body (2) comprises at least one fastening orifice (41) forming an oblique well (42) capable of receiving and guiding, from the outside of said cage (1), a means (43) for fastening the cage (1) to the first vertebral body (4) and/or to the second vertebral body (5).

2. The cage (1) according to the preceding claim, characterized in that said cage body (2) includes a bottom plate (6), the latter is provided with at least one primary through hole (44) through which said fastening means (43) introduced by the fastening orifice (41) can open so as to fasten said cage body (2) to the first vertebral body (4).

3. The cage (1) according to any one of the preceding claims, characterized in that said expansion cap (3) is provided with at least one secondary through hole (45) through which said fastening means (43) introduced by the fastening orifice (41) can open so as to fasten said cage body (2) to the second vertebral body (5), said secondary through hole (45) being configured so as to enable the passage of said fastening means (43) regardless of the angle of inclination of the expansion cap (3) with respect to the bearing surface (6A).

4. The cage (1) according to any one of the preceding claims, characterized in that the inclination stroke of the expansion cap (3) relative to the bearing surface (6A) extends between:
on the one hand, a minimum inclination of said expansion cap (3) in which the bearing element (3A) is at a minimum distance from the bearing surface (6A), and
on the other hand, a maximum inclination of said expansion cap (3) in which the bearing element (3A) is at a maximum distance from the bearing surface (6A).

5. The cage (1) according to any one of the preceding claims, characterized in that the cage body (2) comprises an external envelope (7) having a generally parallelepiped shape, and enclosing a hollow space (E), the external envelope (7) comprising:
said bottom plate (6) comprising the bearing surface (6A),
a longitudinal support wall (9) protruding from the bottom plate (6) up to a support edge (14), the expansion cap (3) being mounted on said support wall (9) of, the cage body (2) via the linking element (12, 13), in the vicinity of the support edge (14).

6. The cage (1) according to the preceding claim, characterized in that the external envelope (7) forms a front wall (10) protruding from the bottom plate (6) up to a stop edge (15), said front wall (10) being disposed opposite the support wall (9), the expansion cap (3) abutting against the stop edge (15) when it is in its minimum inclination so that the inclination stroke of the expansion cap (3) is limited by the stop edge (15).

7. The cage (1) according to the preceding claim, characterized in that the external envelope (7) also forms at least a first transverse wall (11) linking the front wall (10) to the support wall (9), and protruding from the bottom plate (6), the hollow space (E) being formed between the first transverse wall (11), the front wall (10), the support wall (9) and the bottom plate (6).

8. The cage (1) according to claim 6 or 7, characterized in that the bottom plate (6) and/or either one of the walls (9), (10), (11) comprise a plurality of pores (48) which pass therethrough, the passage section of said pores having a characteristic variable comprised between 0.01 mm and 5 mm, said bottom plate (6) comprising an inner surface (38) opposite to the bearing surface (6A), an osteoinductive material being intended to be attached in the hollow space (E) on said inner surface (38), so that a bone fusion could take place between the first vertebral body (4) and the osteoinductive material via said pores (48).

9. The cage (1) according to any one of the preceding claims, characterized in that the expansion cap (3) forms a cap plate with a generally substantially rectangular shape, and delimited by a cap edge (40), the bearing element (3A) being formed by an upper surface (3A) of said cap plate, the linking element (12, 13) extending along said cap edge (40).

10. The cage (1) according to the preceding claim, characterized in that the expansion cap (3) comprises at least one aperture (30) arranged in the latter so as to pass therethrough, so as to enable the introduction of an osteoinductive material through said aperture (30), inside the cage (1).

11. The cage (1) according to any one of the preceding claims, characterized in that said fastening orifice (41) is adapted to enable the connection of a means for introducing and delivering an osteoinductive material, through said fastening orifices (41), inside the cage (1).

12. The cage (1) according to any one of the preceding claims, characterized in that it comprises a hinge forming the linking element (12, 13), and by which the expansion cap (3) is pivotally mounted on the cage body (2), said hinge being formed, on the one hand, by a first hinge element (12) integral with the cage body (2) and, on the other hand, by a second hinge element (13) integral with the expansion cap (3), the first hinge element (12) and the second hinge element (13) being designed to cooperate with each other in order to form the hinge.

13. The cage (1) according to the preceding claim, characterized in that:
the first hinge element (12) comprises a rectilinear groove (34) arranged in the cage body (2) and extending along the axis of inclination (X-X′), the rectilinear groove (34) comprising a retaining flange (35) extending along the axis of inclination (X-X′) over at least part of the length of the rectilinear groove (34),
the second hinge element (13) comprising a rotation rod (36) having an own longitudinal axis, said rotation rod (36) being nested in the rectilinear groove (34) so that said own longitudinal axis is substantially coaxial with the axis of inclination (X-X′), the rectilinear groove (34) being shaped so that the rotation rod (36) could perform a rotation about said axis of inclination (X-X′) relative to the first hinge element (12), the retaining flange (35) allowing retaining the rotation rod (36) within the rectilinear groove (34).

14. The cage (1) according to the preceding claim, characterized in that the second hinge element (13) comprises a tab (37) for fastening the rotation rod (36) to the expansion cap (3), said fastening tab (37) extending from the expansion cap (3) up to the rotation rod (36) over at least most of the length of the rotation rod (36).

15. The cage (1) according to any one of the preceding claims, characterized in that the control means (16) comprises:
at least a first screw (17) for controlling the inclination of the expansion cap (3) operable by a surgeon, and having an own screwing axis (Y-Y′), said first control screw (17) being rotatably mounted about its screwing axis (Y-Y′) either on the cage body (2), or on the expansion cap (3), the first control screw (17) comprising at least a first thread (20) about the screwing axis (Y-Y′) so that the rotation of the screw about its axis (Y-Y′) causes an advance movement of the first thread (20),
the control means (16) also comprising a first transmission means allowing transforming the advance movement of the first thread (20) into an inclination movement of the expansion cap (3) relative to the cage body (2).

16. The cage (1) according to the preceding claim, characterized in that the first transmission means comprises:
at least a first mobile (24) which comprises a threaded orifice (26) by which it is mounted on the first control screw (17), the first thread (20) cooperating with the threaded orifice (26) so as to transmit its advance movement to the first mobile (24) along the screwing axis (Y-Y′),
a first means for blocking in rotation about the screwing axis (Y-Y′) the first mobile (24), so that the latter translates along the screwing axis (Y-Y′) under the action of the rotation of the first control screw (17) about the latter, the first mobile (24) being in contact respectively either with the expansion cap (3), or with the cage body (2), so that its translational movement causes a variation of the inclination of said expansion cap (3).

17. The cage (1) according to the preceding claim, characterized in that the first mobile (24) comprises a first deflection surface (31), the first transmission means comprising at least a first deflection element (28) secured respectively either to the expansion cap (3) or to the cage body (2), and by which the first control screw (17) causes the inclination of the expansion cap (3), the first deflection element (28) and the first deflection surface (31) being in sliding contact on each other, so that said first deflection element (28) is driven by the first deflection surface (31), during the translation of the first mobile (24), in a displacement having at least one non-zero displacement component according to an axis (Z-Z′) orientated in a circular manner with respect to the screwing axis (Y-Y′).

18. The cage (1) according to the preceding claim, characterized in that the first deflection element (28) has a zero displacement component according to the screwing axis (Y-Y′).

19. The cage (1) according to any one of claims 15 to 18, characterized in that the first control screw (17) comprises a second thread (21) about the screwing axis (Y-Y′), the rotation of the first control screw (17) about its screwing axis (Y-Y′) causing an advance movement of the second thread (21) in a direction opposite to that of the first thread (20), the control means (16) comprising a second transmission means allowing transforming the advance movement of the second thread (21) into an inclination movement of the expansion cap (3) relative to the cage body (2), via a second mobile (25), so that the first thread (20) and the second thread (21) contribute together to the control of the inclination of the expansion cap (3) during the rotation of the first control screw (17).

20. The cage (1) according to claim 7 and to any one of claims 15 to 19, characterized in that the first control screw (17) is rotatably mounted on the transverse wall (11) of the cage body (2), so that the screwing axis (Y-Y′) is substantially parallel to the axis of inclination (X-X′) of the expansion cap (3).

21. The cage (1) according to the preceding claim, characterized in that said at least one fastening orifice (41) is arranged in said transverse wall (11) of the cage body (2).

22. A method for manufacturing an expandable intersomatic cage (1) intended to be implanted between a first vertebral body (4) and a second vertebral body (5) of a patient, the method including a step during which:
a cage body (2) comprising a bearing surface (6A) intended to be positioned so as to bear against the first vertebral body (4) is made, an expansion cap (3) comprising an element (3A) for bearing against the second vertebral body (5) is made,
the expansion cap (3) is mounted on the cage body (2) via a linking element (12, 13), so that the expansion cap (3) could pivot according to an inclination stroke about an axis of inclination (X-X′) relative to the bearing surface (6A),
a means (16) for controlling the inclination of the expansion cap (3) capable of maintaining the expansion cap (3) in a desired inclination of the inclination stroke is made.
said method being characterized in that it also comprises a step during which said cage body (2) is provided with at least one fastening orifice (41) forming an oblique well (42) capable of receiving and guiding from the outside of said cage (1) a means (43) for fastening the cage (1) to the first vertebral body (4) and/or to the second vertebral body (5).

23. The method according to the preceding claim, characterized in that the method includes the following steps:
making, on the one hand, a first hinge element (12) integral with the cage body (2),
making, on the other hand, a second hinge element (13) integral with the expansion cap (3),
assembling the first hinge element (12) with the second hinge element (13) in order to form a hinge forming the linking element (12, 13).

24. The method according to the preceding claim, characterized in that:
the first hinge element (12) comprises a rectilinear groove (34) arranged in the cage body (2) and extending along the axis of inclination (X-X′), the rectilinear groove (34) extending between two groove ends (39) at least one of which is open,
the second hinge element (13) comprising a rotation rod (36) having an own longitudinal axis,
the method comprising a step during which the second hinge element (13) is nested on the first hinge element (12) by fitting the rotation rod (36) in the rectilinear groove (34) via the open groove end and by making said rotation rod (36) slide along said rectilinear groove (34) along the axis of inclination (X-X′).

25. The method according to any one of claims 18 to 20, characterized in that at least the step of making the cage body (2) is performed using an additive manufacturing process, preferably a laser melting process.
